(19) United States
(12) Patent Application Publication
(10) Pub. No.: US 2005/0113777 A1
(43) Pub. Date: May 26, 2005

(54) An Absorbent Article Such As A Sanitary Napkin, A Diaper, An Incontinence Guard Or A Panty-Liner

(75) Inventors: Ann Samuelsson, Lindome (SE); Susanne Johansson, Olsfors (SE)

Correspondence Address: BURNS DOANE SWECKER & MATHIS L L P, POST OFFICE BOX 1404, ALEXANDRIA, VA, 22313-1404, US

(73) Assignee: SCA HYGIENE PRODUCTS AB, Goteborgs (SE)

(21) Appl. No.: 10/904,370

(22) Filed: Nov. 6, 2004

Related U.S. Application Data
(60) Provisional application No. 60/523,310, filed on Nov. 20, 2003.

(51) Int. Cl. A61F 13/15; A61F 13/20

(52) U.S. Cl. 604/385.01

(57) ABSTRACT

An absorbent article, such as diapers, sanitary napkins, panty-liners, or incontinence guards for light incontinence, in which an absorption body comprises an absorption sheet. In the longitudinal direction, the absorption sheet is divided into a rear portion, a front portion and a central portion extending between these, the absorbent article comprising an elevation placed on the absorption sheet. The rear portion is divided, in the lateral direction, into two side portions and, extending between these, an open intermediate portion formed by cutting out a fold element from the absorption sheet, the fold element constituting a part of the absorption sheet which is folded over itself and thus forms the elevation.

### CROSS-REFERENCE TO RELATED APPLICATIONS

The present application claims the benefit of U.S. Provisional Application No. 60/523,310, filed in the United States on Nov. 20, 2003, the entire contents of which are hereby incorporated herein by reference.

### FIELD OF THE INVENTION

The present invention relates to an absorbent article such as a sanitary napkin, a panty-liner, or an incontinence guard for light incontinence, including a cover sheet, a backsheet, and an absorption body between these. The absorption body has a longitudinal extent between two short sides and a lateral extent between two long sides. The absorption body includes an absorption sheet which is divided, in the longitudinal direction, into a rear portion, a front portion, and a central portion extending between these, the absorbent article including an elevation placed on the absorption sheet. The invention also relates to a method for producing an absorbent article.

### BACKGROUND

In the production of absorbent articles, manufacturers want a production process which is inexpensive and rapid and yields a good product. The absorbent article can be designed in a number of different shapes, for example an hourglass shape, parallel trapezoid shape, rectangular shape, or similar shapes. Irrespective of which shape is chosen, machines are needed to produce the desired shape. The various parts of the absorbent article are often cut or punched from an original material in the form of a sheet. In the production of the article, it is common for the material left over after production to be discarded. The material left over and discarded represents an unwanted cost to the manufacturer.

It is already known to process sheets of various materials, for example a sheet intended to be used as an absorption body, where the aim is to form an absorbent article with minimal material wastage. For example, it is known to use the left-over material in an absorption body by applying all or part of the left-over material as an additional sheet on the absorption body. The additional sheet can, for example, be applied on that part of the absorption body constituting the so-called wetting point, with the aim of increasing the absorption capacity of the absorbent article. The wetting point is understood as that part of the absorbent article which, during use, is intended to bear against a user's lower abdomen and which is to receive most of the body fluids from the lower abdomen, for example menstruation fluid and urine. The wetting point is often positioned in the central portion of the absorbent article, and there it is customary to construct a raised portion, or what is called a hump, intended to improve the fit of the article against the user's lower abdomen. For example, the hump can be designed in such a way that, during use, it is partially placed between the user's labia to improve the fit of the absorbent article and to improve the absorption capacity of the article.

GB 2195541 discloses a method for producing an hourglass-shaped sanitary napkin, in which an absorption body is provided with a slit on each long side. Each slit runs at a slight angle from the edge of the long side in towards the center. An imaginary fold line runs from a position on the edge of the long side at a distance from the slit to the end of the slit. The slit, the long side and the fold line form a triangle. The triangles are folded over the absorption body in towards the center, where they overlap one another. The folded-over triangles form a hump in the sanitary napkin. A problem with the sanitary napkin according to GB 2195541 is that the upper triangular sheet extends from one edge over the second sheet which extends from the second edge along the surface of the absorption body, which results in distances of different lengths for the two unequal triangles. The difference in distance results in a difference in the extent of the different triangles, resulting in a hump which is asymmetrical and which a user may therefore consider uncomfortable. Another problem which can arise is when the upper triangle is stretched in order to give a symmetrical hump. The stretched upper triangle then pulls harder on its long side edge than the lower triangle pulls on the opposite long side edge. Such uneven distribution of forces can give rise to deformation of the absorption body and, consequently, an asymmetrical absorbent article which provides a poor fit and thus a less comfortable and less reliable product.

U.S. Pat. No. 4,216,773 discloses a method for producing a sanitary napkin designed with a rectangular rear portion, a rectangular front portion and, extending between these, a portion shaped as a parallel trapezoid, i.e., the article has a two-dimensional funnel shape. The absorption body is initially rectangular and slit in the front portion. The slits are arranged substantially parallel starting from one short side. From each long side, a fold line runs to the end point of the nearest slit. The fold line, the slit, a part of the short side and a part of the long side form a fold element in the shape of a rectangle and a triangle. The fold elements are folded overlapping over the fold line across the absorption body. The absorption body thus acquires its funnel shape with the broader portion constituting the rear portion, the narrower portion constituting the front portion, and, extending between these, the parallel trapezium-shaped intermediate portion. The overlapping fold elements form a reinforced absorption zone in the area including parts of the intermediate portion and parts of the front portion. As in GB 2195541, problems arise with the overlapping fold elements on account of asymmetry.

Thus, there is still a first requirement for an improved production of an absorbent article with a minimum of waste, and a second requirement for an absorbent article with good absorption properties and a good fit produced by a method which results in minimum waste.

### SUMMARY

The present invention is aimed at solving the problems, indicated above in the prior art, in respect of an absorbent article such as a sanitary napkin, a panty-liner, or an incontinence guard for light incontinence. The present invention solves the problems by means of an absorbent article comprising a cover sheet, a backsheet, and an absorption body between these. The absorption body comprises an absorption sheet with a longitudinal extent between two short sides and a lateral extent between two long sides. The absorption sheet is divided, in the longitudinal direction, into a rear portion, a front portion, and a central portion extending between these. The absorbent article additionally comprises an elevation placed on the absorption sheet. The elevation is intended to be placed towards the user's genitals during use of the absorbent article. The advantages of using an elevation in absorbent articles is already known, see for example WO 98/22062. For example, the elevation provides a good fit against the user's genitals and in this way reduces the risk of leakage and thus gives the user an increased sense of protection.

The invention is characterized in that the rear portion is divided, in the lateral direction, into two side portions and, extending between these, an open intermediate portion formed by cutting out a fold element from the absorption sheet, the fold element constituting a part of the absorption sheet which is folded over itself and thus forms the elevation.

The expression “open” intermediate portion signifies that a recess has been made in the absorption sheet. The open intermediate portion in the absorbent article can be filled with an absorption material or other suitable material. The open intermediate portion does not therefore have to be open in the sense that it is empty or that it forms a hollow in the absorbent article.

The elevation advantageously includes a fold element forming part of the absorption sheet being folded over along a lateral fold line which coincides with a laterally extending first edge of the open intermediate portion. The elevation is also advantageously placed substantially on the central portion of the absorption sheet.

The absorbent article is advantageously produced by a method comprising the following steps:
a first slit and second slit are formed in the rear portion in such a way that the rear portion is divided, in the lateral direction, into two side portions and, extending between these, a portion which forms a fold element, and;
the fold element is then folded over the absorption sheet in such a way that the fold element forms an elevation on the absorption sheet.

The fold element is advantageously folded over the absorption sheet along a lateral fold line in such a way that the elevation is formed and that this folding-over results in formation of the open intermediate portion between the two side portions, and the fold line coincides with the laterally extending first edge of the open intermediate portion.

An advantage of the invention is that the absorbent article acquires an elevation, with the advantages indicated above, and at the same time the method results in minimum waste, in fact no waste. The method according to the invention also advantageously affords an elevation which is symmetrical around the longitudinal center line of the absorbent article. Another advantage of the invention is that the divided rear portion is formed without waste. A divided rear portion advantageously gives an absorbent article which is flexible, conformable and secure. The divided rear portion follows the body better than an undivided rear portion, and the absorbent article therefore tends not to be deformed in an undesirable way when a user moves. U.S. Pat. No. 5,514,104 and SE 518364, for example, indicate a number of embodiments of an absorbent article comprising an absorption sheet with divided rear portion, and the advantages associated with such an article.

The slits can be formed by cutting, punching, sawing, machine-cutting, laser, or any other known technique for making slits in a sheet of material.

According to one embodiment of the invention, the slitting is carried out in two substantially parallel slits in a substantially longitudinal direction, in such a way that the design of the fold element is substantially rectangular, and the first edge constitutes one short side of the fold element. The lateral fold line thus coincides with that part of the rectangle which constitutes one short side of the rectangle. The advantage of parallel slits is that, from the point of view of production technology, it is easy to make two parallel slits.

According to another embodiment of the invention, the slitting is carried out in two substantially diagonal slits from the short side of the rear portion to the fold line, in such a way that the design of the fold element is substantially in the form of a parallel trapezoid comprising a longitudinally extending rectangular central part flanked on both sides by angled parts, and the first edge constitutes the shorter of the two parallel sides of the parallel trapezoid. The lateral fold line thus coincides with that part of the parallel trapezoid which constitutes the shorter of the two parallel sides of the parallel trapezoid. The angled parts are preferably triangular. The advantage of diagonal or oblique slits is that an absorbent article is easier to fold at the time of use. What is intended by folding here is that an absorbent article is normally deformed during use. The deformation will preferably take place along a centrally positioned and longitudinally extending fold line. When the article folds over the fold line, advantageous adaptation to the user's lower abdomen takes place. In those cases where the open intermediate portion is designed as a parallel trapezoid in accordance with the above, the diagonal edge portions in the open intermediate portion point in towards the centrally positioned and longitudinally extending fold line. The absorbent article then has an improved ability to be folded along the centrally positioned and longitudinally extending fold line.

According to one embodiment of the invention, the angled parts of the fold element are folded in between the absorption sheet and the central part of the fold element, along longitudinally extending parallel fold lines, in such a way that the elevation acquires a substantially rectangular or triangular profile seen in perspective view from above. The elevation thus consists here of the fold element's folded-in angled parts and the central part. The angled parts can be folded in between the absorption sheet and the central part of the fold element several times, i.e., almost a rolling-up of the angled parts in towards the longitudinal center line.

According to another embodiment of the invention, the central part of the fold element is folded in two over a centrally positioned longitudinal fold line and the angled parts of the fold element are unfolded across the absorption sheet substantially at right angles to the double-folded central part. The elevation thus includes the fold element's central part folded in two over a centrally positioned longitudinal fold line, with the fold element's angled parts unfolded across the absorption sheet substantially at right angles to the double-folded central part, in such a way that the elevation acquires a profile in the form of a cone with brim.

According to another embodiment of the invention, the slitting is carried out in two substantially diagonal slits from the short side of the rear portion to the fold line, in such a way that the design of the fold element is substantially in the form of a parallel trapezoid comprising a longitudinally extending rectangular central part flanked on both sides by angled parts, and the first edge constitutes the longer of the two parallel sides of the parallel trapezoid.

According to one embodiment of the invention, the elevation is formed by means of the fold element being folded in two on itself along a laterally extending fold line. In this embodiment, the elevation thus includes the fold element folded in two on itself. The embodiment is especially advantageous in those cases where the fold element is designed as a rectangle or parallel trapezoid and is not folded in another way.

According to a further embodiment of the invention, the elevation includes the fold element rolled up in its longitudinal extent.

Since the fold element is formed from the absorption sheet via the two slits, the design of the fold element corresponds to the design of the intermediate portion when the absorbent sheet is in the unfolded position. It should be noted, however, that the open portion corresponds only to that part of the fold element which is folded over the absorption sheet. The slits can thus be made longer than the sides in the open intermediate portion, i.e., the intermediate portion of the rear portion can have a longer longitudinal extent than the fold element. This means that the lateral fold line always coincides with the one edge of the open intermediate portion and can cross the slits when the slits are longer than the longitudinal extent of the intermediate portion.

According to one embodiment of the invention, the elevation comprises a stiffening element which strengthens the shape of the elevation. The stiffening element can include any suitably rigid material which is liquid-absorbing. Such a liquid-absorbing material may, for example, be a compressed material of the kind the absorption body or the absorption sheets is made of. A stiffening element can additionally be made of a liquid-permeable material, for example a plastic film formed with holes. There are therefore a great many techniques for obtaining a shape-stable elevation. Reference may be made to WO 98/22062, for example, in which an absorbent article is equipped with a shape-stable elevation.

According to one embodiment of the invention, the absorption sheet comprises a sheet of a stiffening material, the stiffening sheet being cut during production of the absorbent article and being folded together with the absorption sheet. In another embodiment of the invention, the fold element can be compressed by suitable means to form a stiffening element, for example at the same time as the fold element is cut or punched.

The absorbent article can additionally be equipped with elastic elements which are arranged around the elevation with the aim of strengthening the shape of the elevation. The elastic elements can additionally be used for shaping the absorbent article in a suitable way, for example a cup-shaped front portion or a ridge-like elevation in the central portion. Such elastic elements are mentioned, inter alia, in SE 518364.

According to one embodiment of the invention, the slits are formed by the absorption sheet being folded in two along a longitudinally extending center line, after which the slits are produced by making a cut through the double-folded absorption sheet. After the slits have been made, the absorption sheet is unfolded into a flat state and the fold element is then folded over the absorption sheet.

An advantage of this method is that the double-folded element has, by being folded in two, acquired a fold indication which gives the fold element an angled appearance. The advantage lies in the fact that the fold element is not folded back to a flat state, but instead the fold element in its substantially double-folded state is folded over the absorption sheet in such a way that the fold indication present in the fold element coincides with the top part of the elevation.

The absorbent article is formed by the absorption body being applied between the backsheet and the cover sheet. The absorption body can consist only of the worked absorption sheet, but the absorption sheet can additionally be included as a sheet in the absorption body. The absorption body can thus include several sheets of absorption material of different types, where the worked absorption sheet constitutes a sheet with an elevation.

In all the abovementioned embodiments, the fold element, in the state when folded over, can be secured to the rest of the surface of the absorption sheet by a suitable method, for example by gluing (preferably hydrophilic types of glue), mechanical securing elements (for example velcro), or welding, such as ultrasound, heat, or the like.

The various types of folding of the fold elements which are described above can be carried out by known methods, for example machines comprising tab fingers, or similar devices.

The techniques described above are used to create an elevation on that part of the absorption sheet which faces the cover sheet, i.e., the top side. This can be done by the fold element, in any of the above embodiments, being folded over on the top side, but it can also be obtained by the fold element being folded over on the underside of the absorption sheet. In the latter case, the elevation is formed by the fold element pressing against the underside of the absorption sheet in such a way that the absorption sheet is deformed and in this way forms the elevation.

The backsheet advantageously constitutes a liquid barrier and includes a liquid-impermeable material. Thin, liquid-tight plastic films are suitable for the purpose, but it is also possible to use material which is liquid-permeable to start with but which has been provided with a coating of plastic, resin, or other liquid-tight material. This prevents leakage of liquid from the underside of the absorbent article. The barrier sheet can therefore include any material which satisfies the criterion of liquid-impermeability and has sufficient flexibility and skin-friendliness for the purpose. Examples of materials which are suitable as barrier sheets are plastic films, nonwovens, and laminates thereof. The plastic film can, for example, be of polyethylene, polypropylene or polyester. The barrier sheet can alternatively include a laminate of a liquid-impermeable plastic sheet, facing towards the absorption body, and a nonwoven, facing towards the user's underwear. Such a construction provides a leaktight barrier sheet with a textile feel. The liquid-blocking backsheet can also be made of a material permeable to vapour. A breathable backsheet of this kind can, for example, be made of what is called SMS (spunbond-meltblown-spunbond) material or a breathable plastic film including polyethylene. A plastic film of this kind is described in EP 283 200. To maintain the breathability even when the material has been applied to a product, the underside of the product should not be completely covered by securing elements.

The cover sheet can be made of any conventional material which is liquid-permeable and skin-friendly, for example a nonwoven, a perforated plastic film, or a laminate of a perforated plastic film and a nonwoven.

The absorption body is preferably produced from one or more layers of cellulose pulp. The pulp can originally be in the form of rolls, bales or sheets which, in the production of the sanitary napkin, are dry-defibred and converted in fluffed form to a pulp mat, sometimes with admixture of what are called superabsorbents, namely polymers with the capacity to absorb several times their own weight of water or body fluid. An alternative to this is to dry-shape a pulp mat as is described in WO 94/10956. Examples of other absorption materials that can be used are various types of natural fibers such as cotton fibers, peat, or similar natural fibers. It is of course also possible to use absorbent synthetic fibers, or particles of a high-absorbency polymer material of the type which on absorption chemically binds large quantities of liquid, with formation of a liquid-containing gel, or to use mixtures of natural fibers and synthetic fibers. The absorption body can also contain other components, such as shape-stabilizing elements, liquid-spreading elements, or binders such as thermoplastic fibers which have been heat-treated in order to hold short fibers and particles together in a coherent unit. It is also possible to use different types of absorbent foam material in the absorption body. However, the absorption sheet must be of such a type that can be shaped by cutting and folding. It may therefore be expedient to add binding fibers to the above-described absorption materials in order to obtain a suitable absorption sheet.

The absorption sheet can additionally comprise a support sheet, for example made of a perforated plastic material and intended to support the absorption material. The support sheet can additionally constitute a stiffening material which provides an absorption sheet stiffer than the surrounding sheets. Such a stiffening sheet is additionally expedient if the elevation is to be given an increased shape stability.

A securing device is preferably arranged on the backsheet of the absorbent article and is intended, during use, to secure it to a user's briefs. The securing device preferably includes glue, but it can also include a mechanical securing device such as velcro, press-studs, friction coatings, clip elements or similar securing devices. The glue can be applied in one or more strands or in some other pattern. Alternatively, the whole of the underside of the absorbent article is coated with glue. It is also possible to use a securing glue which is able to breathe and apply it across the whole backsheet of the article so that it functions as a combined barrier sheet and securing device.

A protective sheet is advantageously applied on the securing device. The protective sheet is preferably a silicone-treated paper, but other types of protective sheet are of course also possible, for example, waxed paper, stamped or release-agent-treated plastic film, textile strips to attach to velcro, or similar protective sheets.

The height of the elevation above the surface of the absorbent article must be adapted so that good contact between the user's body and the elevation is guaranteed during use. In the case of hard or stiff elevations, the elevation is essentially unaffected by the forces to which it is exposed during use. It is important then to ensure that the elevation is not so high that it presses against the user's body and thus causes discomfort during use. It is also necessary to ensure that the elevation does not chafe the sensitive soft parts at the user's crotch. It has been found that an elevation which, at its highest part, protrudes from the surface of the article by a distance corresponding to the thickness of the absorption sheet, but by not more than 20 mm, satisfies the requirements in terms of good contact with the body and a high degree of comfort for the user.

An elevation in accordance with the invention is relatively narrow, expediently between 2 mm and 25 mm at the base, and preferably between 12 mm and 16 mm. At the top, the elevation is expediently between 3 mm and 10 mm wide, and preferably between 4 mm and 6 mm. The elevation is formed with an essentially triangular cross section and is thus broader at the base than at the top. As the elevation is relatively narrow, it can protrude slightly between the user's labia without causing the user discomfort. In this connection, it is an advantage if the elevation parts slightly from the labia, since the transfer of liquid from the user to the article is thus made easier.

At the rear part of the elevation, the latter should have a shape such that it bears in close contact with the user's body, in the area behind the vaginal opening. This prevents body fluid from running rearwards in the furrow between the user's buttocks and leaking out of the article. As has been mentioned earlier, such rearward leakage is especially problematic when the user is lying down. Correspondingly, the front part of the elevation should match the shape of the user's body in the area in front of the vaginal opening.

It is important that the body fluid excreted is immediately collected and absorbed into the elevation. This prevents fluid from gathering between the user's body and the elevation. When the user stands up or moves in some other way, there is in fact a risk of a gap forming in the front part of the elevation, between the elevation and the user's body. If a large amount of unabsorbed fluid has been trapped between the user's body and the surface of the article, such fluid can flow out through the gap. Such sudden flows of fluid are obviously undesirable, on the one hand because they are unpleasant for the user and, on the other hand, because they considerably increase the risk of leakage.

To achieve optimum contact between the elevation and the user's body, the length of the elevation is also of importance. The elevation should have a shape and an extent along the article such that the elevation bears in tight contact against the skin behind the user's vaginal opening.

In addition, a raised edge barrier can advantageously be arranged on both sides of the central elevation. The edge barriers increase the leaktightness of the article by acting as seals against the user's groin. Raised edge barriers can be obtained in a number of different ways. The edge barriers are preferably made of the same material as the central elevation and thus have substantially the same resistance to deformation as the latter. However, it is possible to imagine other types of edge barriers, such as elastic side edges, foam rubber strips, wadding, or the like. The edge barriers can either be purely physical barriers or can prevent passage of fluid by absorbing body fluid.

The embodiments given above have been described in connection with the rear portion, central portion and front portion of the absorption sheet. The rear portion can have a longitudinal extent which corresponds to one third to one half of the extent of the absorption sheet in the longitudinal direction. In those cases where the rear portion constitutes half of the extent of the absorption sheet, the fold element will also have an extent corresponding to half the length of the absorption sheet. The folded-over fold element thus extends over essentially the whole part of the absorption sheet not including the rear portion. The remaining half of the absorption sheet can be divided up into a front portion and a rear portion depending on the design of the article. A rectangular absorption sheet can, for example, be regarded as missing a central portion, for which reason the front half of the absorption sheet can be regarded as comprising only a front portion or only a central portion. In the case of an hourglass-shaped absorption sheet, by contrast, the front half can be regarded as comprising a central portion and a front portion, the fold element in the embodiment extending over both the central portion and the front portion.

It should be noted, however, that the division of the absorption sheet into portions is not to be understood as meaning that there are sharp lines between the different portions, and instead the division is primarily intended to facilitate the description of the absorbent article with reference to the differences which exist between the different portions depending on how they are to be placed in relation to the user's body. The transition between the different portions in the present embodiment thus does not take place on fixed lateral lines, but instead within transition areas. The fold element can, for example, be formed by slits formed in both the rear portion and the central portion, in which case the fold element is folded over both the remaining part of the central portion and the front portion. Regardless of the definition of the rear portion, the central portion and the front portion, the fold element will be formed and folded in such a way that the absorption sheet acquires an elevation which, during use of the absorbent article, will constitute the wetting point, i.e., will bear against the user's genitals. The elevation can then extend over the absorption sheet by a distance in the longitudinal direction representing one quarter to one half of the longitudinal extent of the absorption sheet.

The central portion constitutes that part of the absorbent article which, during use, is intended to receive and absorb most of the fluid emitted to the absorbent article, namely the so-called wetting point. As has been mentioned above, the elevation will constitute at least the wetting point and is consequently placed essentially in the central portion according to the terminology used in the application.

### BRIEF DESCRIPTION OF THE FIGURES

The invention will be described below with reference to a number of figures, where:

FIG. 1 is a diagrammatic illustration showing a top view of an absorption sheet according to a first embodiment of the invention, with a fold element in the unfolded position;

FIG. 2 is a diagrammatic illustration showing a top view of an absorption sheet according to the first embodiment of the invention, in which the fold element is in a folded-over position;

FIG. 3 is a diagrammatic illustration showing a side view along one long side of the absorption sheet according to FIG. 2;

FIG. 4 is a diagrammatic illustration showing an absorbent article comprising an absorption sheet according to FIG. 2;

FIG. 5 is a diagrammatic illustration showing a top view of an absorption sheet according to a second embodiment, the fold element being in a folded-over position;

FIG. 6 is a diagrammatic illustration showing a cross section along the line A-A from FIG. 5, the fold element being folded together according to one embodiment;

FIG. 7 is a diagrammatic illustration showing a cross section along the line A-A from FIG. 5, the fold element being folded together according to a second embodiment;

FIG. 8 is a diagrammatic illustration showing a cross section along the line B-B from FIG. 5, the fold element being folded together according to another second embodiment;

FIG. 9 is a diagrammatic illustration showing a side view along one long side of the absorption sheet according to FIG. 7 or FIG. 8;

FIG. 10 is a diagrammatic illustration showing a top view of an absorbent article comprising an absorption sheet according to a third embodiment of the invention, in which fold element is in a folded-over position; Page 16 of 47

FIG. 11 is a diagrammatic illustration showing a top view of an absorbent article comprising an absorption sheet according to a fourth embodiment of the invention, the fold element being in a folded-over position;

FIG. 12a is a diagrammatic illustration showing a folded-up absorption sheet in a method for slitting the absorption sheet;

FIG. 12b is a diagrammatic illustration showing a perspective view of FIG. 12b, and

FIG. 12c shows the absorption sheet according to FIGS. 12a and 12b in a deployed position after slitting.

### DETAILED DESCRIPTION OF THE PREFERRED EMBODIMENTS

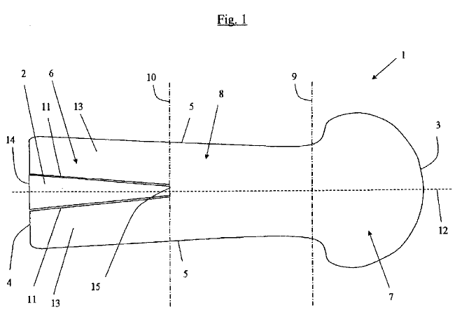

FIG. 1 is a diagrammatic illustration showing a top view of an absorption sheet 1 according to a first embodiment of the invention, with a fold element 2 in the folded-out position. The absorption sheet 1 has a longitudinal extent between two short sides 3, 4 (a front edge 3 and a rear edge 4) and a lateral extent between two long sides 5. In the longitudinal direction, the absorption sheet 1 is divided into a rear portion 6, a front portion 7, and a central portion 8 extending between these. FIG. 1 shows a front, laterally extending broken line 9 which defines the border between the front portion 7 and the central portion 8, and a rear, laterally extending broken line 10 which defines the border between the central portion 8 and the rear portion 6. The absorption sheet 1 has an asymmetrical design in the form of a parallel trapezoid whose short parallel sides merge into a semicircle.

FIG. 1 shows two slits 11 formed in the rear portion, which slits 11 have an extent from the short side 4 (rear edge) of the rear portion 6 to the rear broken line 10, i.e., to the central portion 8. The slits 11 extend from the rear edge 4 diagonally in towards a center line 12 extending longitudinally in the absorption sheet. The slits 11 thus converge in the direction towards the central portion 8 but never intersect one another. The slits 11 interrupt the rear broken line 10 at a predetermined distance from the longitudinally extending center line 12. In the lateral direction, the two slits 11 divide the rear portion 6 into two side portions 13 and, extending between these, the fold element 2. In FIG. 1, the fold element 2 is formed substantially as a parallel trapezoid, where the diagonal sides constitute the slits 11 and where the longer of the parallel sides 14 of the parallel trapezoid constitutes part of the rear edge 4 and where the short side 15 of the parallel trapezoid coincides with the rear broken line 10.

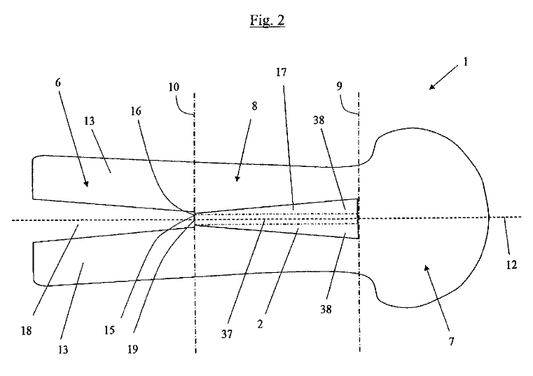

FIG. 2 is a diagrammatic illustration of a top view of an absorption sheet 1 according to the first embodiment of the invention, where the fold element 2 is in a folded-over position. Folded-over position here means that the fold element 2 has been folded over the central portion 8 of the absorption sheet 1 from its deployed position in FIG. 1, along a lateral fold line 16 which coincides with the rear broken line 10. The lateral fold line does not have to coincide with the rear broken line 10, and instead the lateral fold line 16 can be placed at any suitable point in the rear portion 6. The design of the fold element 2 in the shape of a parallel trapezoid is divided up principally into a longitudinally extending rectangular central part 37 flanked by angled parts 38 on both sides.

The fold element 2 forms, on the absorption sheet 1, an elevation 17 placed on the central portion 8 of the absorption sheet 1. In FIG. 2, the rear portion 4 is divided in the lateral direction into the two side portions 13 and, extending between these, an open intermediate portion 18. The open intermediate portion 18 corresponds in shape to the shape of the fold element 2 in FIG. 1. The elevation 17 is thus formed by the fold element 2 constituting a part of the absorption sheet 1 which is folded over itself along the lateral fold line 16 coinciding with a laterally extending first edge 19 of the open intermediate portion 18. The lateral fold line 16 coincides with the first edge of the open intermediate portion 18 regardless of how the fold element is folded. An example that may be mentioned is when the fold element 1 is folded along the lateral fold line 16 in a position between the start and end of the slits 11, i.e., somewhere along the extent of the slits 11, which in the figure is between the rear edge 4 and the rear broken line 10.

In the present embodiment, the absorption sheet 1 includes the semicircle-shaped front portion 7, the central portion 8 in the shape of a parallel trapezoid, and the rear portion 6 including the two side portions 13. The absorption sheet 1 according to the described embodiment of the invention is designed with a front portion 7 which is wider than the central portion 8, and with a central portion 8 which is narrower than both the front portion 7 and the rear portion 6.

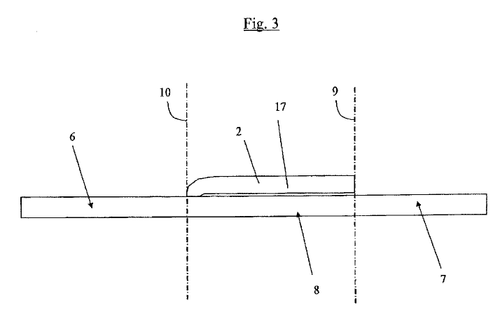

FIG. 3 is a diagrammatic illustration showing a side view along one long side 5 of the absorption sheet 1 according to FIG. 2. In FIG. 3, the fold element 2 is shown folded over the central portion 8 of the rest of the absorption sheet 1. FIG. 3 shows that the fold element 2 extends over the whole of the central portion 8, i.e., from the rear broken line 10 to the front broken line 9. The fold element 2 can of course be formed in such a way that it extends only partially over the central portion 8, depending on the length of the slits 11 and/or on the size of the central portion 8, and depending on where the lateral fold line is placed.

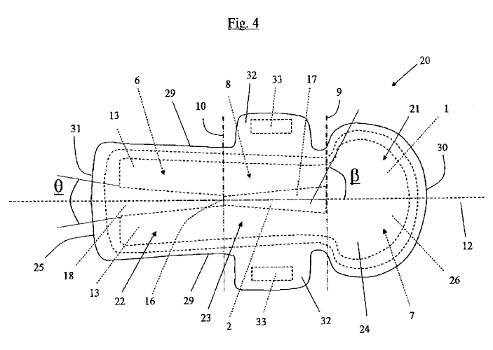

FIG. 4 is a diagrammatic view of an absorbent article 20 comprising an absorption sheet 1 according to FIG. 2. A first transverse line 9 in FIG. 4 coincides with the front broken line 9 in FIGS. 1-3, and a second transverse line 10 coincides with the rear broken line 10 in FIGS. 1-3. The absorbent article 20 is thus divided into a front portion 21, a rear portion 22, and a central portion 23 extending between these, which portions correspond to the portions 6-8 of the absorption sheet indicated in FIGS. 1-3. The absorbent article 20 comprises a liquid-permeable cover sheet 24 arranged on that side of the absorbent article 20 which, during use, is intended to face towards the user, a liquid-tight backsheet 25 arranged on that side of the absorbent article 20 which during use is intended to face away from the user, and an absorption body 26 enclosed between the cover sheet 24 and the backsheet 25.

As has been mentioned above, the division of the absorption sheet 1 and the absorbent article 20 into the front portions 7, 21, the rear portions 6, 22, and the central portions 8, 23 is not to be understood as meaning that there are sharp lines between the different portions. The division is primarily intended to facilitate the description of the absorbent article with reference to the differences which exist between the different portions depending on how they are interned to be placed in relation to the body of the user. Therefore, the transition between the different portions in the present embodiment does not take place at fixed transverse lines, but instead within transition areas which, according to FIG. 4, are situated at a distance of about one quarter to one third of the length of the absorption sheet from the front edge and rear edge, respectively, of the absorption sheet. In FIG. 4, the previously mentioned wetting point is situated in the central portion 23 of the absorbent article 20.

The absorption body 26 can advantageously comprise different sheets of different types of absorbent material. FIG. 4 shows an absorption body 26 comprising two different sheets of absorbent material, of which one sheet constitutes the absorption sheet 1 according to the invention, and where the second sheet constitutes a second absorbent sheet 27. In FIG. 4, the absorption sheet 1 is arranged between the backsheet 25 and the second absorbent sheet 27, and the second absorbent sheet 27 is arranged between the absorption sheet 1 and the cover sheet 24. The second absorbent sheet 27 is advantageously a “fast” material which transports fluids efficiently, and the absorption sheet 1 has a good capacity for storing fluids. However, depending on the choice of material of the different absorbent sheets, the absorption sheet 1 according to the invention can be placed between the cover sheet 24 and the second absorbent sheet 27, and the second absorbent sheet 27 can be placed between the absorption sheet 1 and the backsheet 25.

The backsheet 25 and the cover sheet 24 are connected to one another outside the absorption body 26 and form a protruding edge 28 around the entire periphery of the absorbent article 20. The cover sheet 24 nd the backsheet 25 can be joined together in any suitable manner, for example by gluing, stitching or welding, either by heat or by ultrasound.

To obtain an absorbent article 20 which provides a good fit and is comfortable for the user, it is necessary for the absorbent article to have a design which to a very great extent is adapted to the user's anatomy. It is therefore of especially great importance that the width of the absorbent article 20, at least in the front part of the central portion 23, does not exceed about 40 mm. The front portion 21 is advantageously wider than the central portion 23 so that the wider front portion 21 together with the narrower central portion 23 shapes itself around the user's body and to a certain extent “hooks” the absorbent article against the user's legs, thus making it possible for the absorbent article 20 to remain in place against the user's body. The wider front portion 21 preferably has a slightly oval shape. One reason for this is that an oval-shaped front portion 21, when it is in use, is bent inwards and a cup shape is formed, i.e., the upward bending of the front portion, in relation to the central portion, does not take place along a sharply defined fold line, and instead the curvature is continuous in the longitudinal direction of the absorbent article. In this way, the front portion 21 forms a gently rounded cup, which adapts extremely well to the user's anatomy. In addition, the oval shape of the front portion 21 also provides a large absorption area/volume for the absorption body 26.

The absorbent article 20 according to FIG. 4 also has two longitudinal long sides 29 (side edges), a transverse, concavely curved short side 30 (front edge), and a transverse, convexly curved short side 31 (rear edge). In FIG. 4, the absorbent article is shown with so-called wings 32, i.e., foldable elements which, during use of the absorbent article 20, are intended to be folded over the side edges of a user's briefs. The wings 32 are placed on the central portion 23 of the absorbent article 20, along the side edges 29. The wings 32 are equipped with securing device 33 which are used to secure the wings on the outside of the briefs when the wings have been folded over the briefs. Suitable securing devices 33 are glue, velcro, and the like. However, the absorbent article 20 does not need to be equipped with wings 32.

The absorbent article 20 is designed in such a way that the first transverse line 9, which extends in the transverse direction of the absorbent article, intersects the side edges 29 of the absorbent article. At the first transverse line 9, the inclination of the side edges 29 in relation to the longitudinal center line 12 changes, as a result of which the width of the absorbent article increases in the direction towards the front edge 30, by which means the front portion 21 has a maximum width exceeding the width of the central portion 23 at the first transverse line 9. The maximum width of the front portion 21 is suitably at least twice the width of the central portion 23 at the first transverse line 9. The inclination of the side edges 29 at the front portion 21 is defined by an angle β between each respective side edge 29 and a longitudinal line which is parallel to the center line 12, as a result of which β is between 30° and 90° and the width of the central portion 23 of the absorption sheet 1 at the first transverse line 10 is between 15 and 45 mm and preferably between 20 and 40 mm. All of the data mentioned above are considered to apply to the whole of the absorbent article 20, but this is true only if the outer edge 28 of the absorbent article 20 almost follows the outer edge of the absorption body 26. The absorption sheet 1 according to FIGS. 1-3 thus has a shape which largely corresponds to the absorbent article 20 according to FIG. 4, except for the wings 32.

The absorption sheet 1 according to the invention is advantageously stiffer than the second absorbent sheet 27. However, the material of the absorption sheet 1 does not have to be stiffer than the second absorbent sheet 27 per se, for example if the absorption sheet 1 is produced in such a way that it is stiffer than the second absorbent sheet 27, for example by adhesive lamination of several sheets of identical or different materials or by another suitable production alternative, or if the absorption sheet is equipped with a stiffening element. The absorption sheet 1 according to the present invention advantageously serves as a shaping element, as a result of which the area of the absorption sheet lying substantially within the area of the rear portion 6 is divided into branches, i.e., the two side portions 13. The two branches 13 form an angle θ at the point where the branches divide. The angle θ between the branches is between 10° and 120°, preferably between 15° and 40°, and the length of the branches is between 20 and 350 mm, preferably between 50 and 150 mm. As has been mentioned above, the second transverse line 10 coincides with the rear broken line 10 in FIG. 2, i.e., is placed in the imagined area where the rear portion meets the central portion.

The two branches 13 here include the stiffer absorption sheet and are covered by the second absorbent sheet 27. The absorbent article 20 can additionally be equipped with elastic members (not shown). Advantages of a divided rear portion 6 are described in U.S. Pat. No. 5,514,104, for example, and advantages of a divided rear portion 6 together with an elastic member are well described in SE 518364.

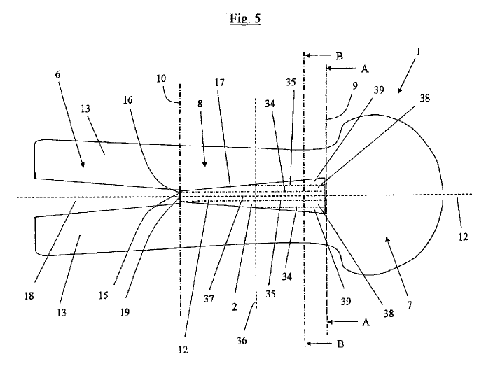

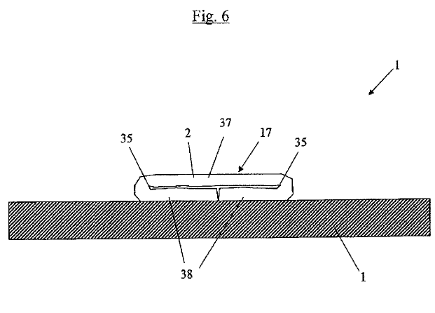

FIG. 5 shows a diagrammatic top view of an absorption sheet 1 according to a second embodiment of the invention, where the fold element 2 is in a folded-over position. FIG. 5 shows a cross section line A-A which coincides with the front broken line 9, and a cross section line B-B parallel with the cross section line A-A, but offset towards the rear portion 6. The fold element 2 in FIG. 5 comprises a number of longitudinally extending fold lines. The fold lines include an outer pair of fold lines 34 and an inner pair of fold lines 35. FIGS. 6-8 show a number of embodiments of the formation of the elevation 17 with different ways of folding the fold element 2. The fold lines 34, 35 in FIG. 5 will be used, in connection with FIGS. 6-8, to describe the different possible ways of folding the fold element 2. FIG. 5 additionally shows a centrally positioned and longitudinally extending fold line 40. FIG. 5 also shows that the angled parts 38 comprise outer fold parts 39.

A laterally extending fold line 36 is also shown in FIG. 5. According to one embodiment of the invention, the fold element 2 can be folded in two over the lateral fold line 36. This embodiment is not shown.

FIG. 6 shows a diagrammatic cross section along the line A-A in FIG. 5, where the fold element 2 is folded up according to one embodiment. The elevation 17 here includes the central part 37 of the fold element 2 and the angled parts 38 folded in between the absorption sheet 1 and the central part 37 along longitudinally extending parallel fold lines, in such a way that the elevation 17 acquires a substantially rectangular profile seen in perspective from above. In the present embodiment, the longitudinally extending fold lines can include either the inner pair of fold lines 35 or the outer pair of fold lines 36.

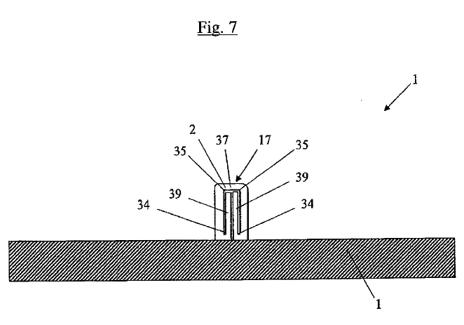

FIG. 7 shows a diagrammatic cross section along the line A-A in FIG. 5, where the fold element 2 is folded up according to a second embodiment. The elevation 17 here includes the central part 37 of the fold element 2 and the angled parts 38 folded in between the absorption sheet 1 and the central part 37. The angled parts 38 comprise outer fold parts 39 which are folded in two over the outer pair of fold lines 34. The angled parts 38 folded in two are then folded ca. 90° over the inner apir of fold lines 35, in such a way that the outer fold parts 39 are folded into the elevation 17 with the tp faces of the outer fold parts 39 against one another. The elevation 17 thus acquires a substantially rectangular profile seen in perspective from above. The outer fold parts 39 of the fold element 2 form shape-strengthening strengthening elements for the elevation 17. The elevation 17 can also be equipped with other shape-strengthening material, for example a compressed fiber product which gives the desired shape-strengthening.

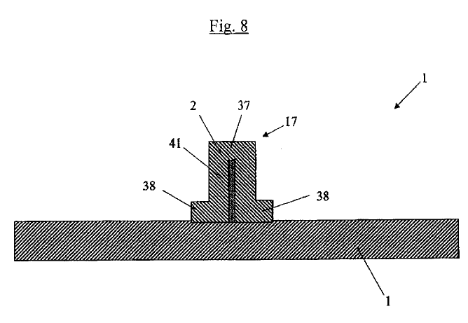

FIG. 8 shows a diagrammatic cross section along the line B-B in FIG. 5, where the fold element 2 is folded up according to another second embodiment, where the elevation includes the central part 37 of the fold element folded in two over the longitudinal fold line 40 placed centrally in FIG. 5, and the angled parts 38 of the fold element 2 are folded out against the absorption sheet 1 substantially at right angles to the central part 37 folded in two, in such a way that the elevation 17 acquires a profile in the form of a cone with brim. The angled parts in FIG. 8 are folded along the inner pair of fold lines 35 in FIG. 5, but they can also be folded along the outer pair of fold lines 34. Such a cone with brim can be likened to a witches' hat in profile. FIG. 8 shows that the elevation 17 comprises a shaping element or stiffening element 41 which corresponds to the desired profile of the elevation. The stiffening element 41 can be made of an absorbent material which has been compressed, for example, a fiber material, or of a nonabsorbent material such as plastic equipped with holes to permit transport of fluid through the elevation.

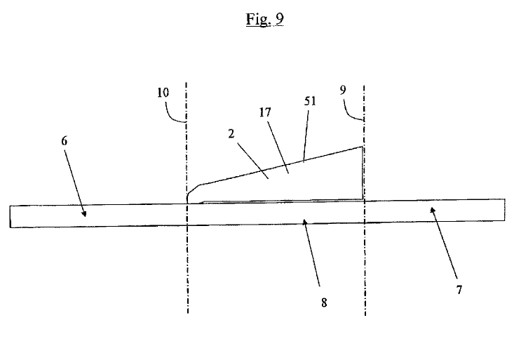

FIG. 9 shows a diagrammatic side view along one long side of the absorption sheet according to FIG. 7 or FIG. 8. A common feature of both embodiments is the triangular profile of the elevation 17. Another profile will of course be obtained by deforming the triangular elevation 17 as shown in FIG. 9.

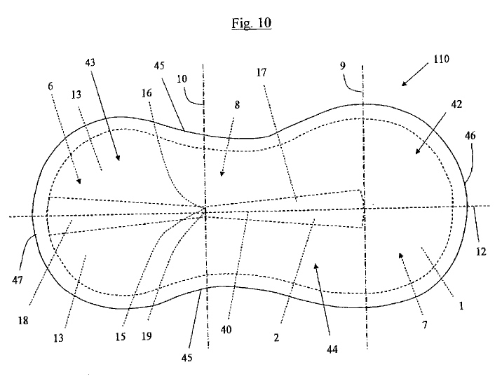

FIG. 10 shows a diagrammatic top view of an absorbent article 110 comprising an absorption sheet 1 according to a third embodiment of the invention, where the fold element 2 is in a folded-over position. The difference between this embodiment and the embodiments shown in FIGS. 1-9 is that the absorption sheet 1 is hourglass-shaped instead of asymmetrical. The absorption sheet 1 thus has a front part 42, intended to be directed to the front on the user during use, a rear part 43, intended to be directed to the rear on the user during use, and an intermediate, narrower middle part 44 intended to be applied in the user's groin region. In addition, the absorbent article has two concavely curved side edges 45, a convexly curved front edge 46, and a similarly convexly curved rear edge 47. All that has been said concerning the formation of the elevation 17 in connection with the embodiments shown in FIGS. 1-9 applies to the embodiment in FIG. 10, where the previously mentioned rear portion has been replaced by the rear part mentioned here. In the same way, the central portion is replaced by the middle part, and the front portion by the front part.

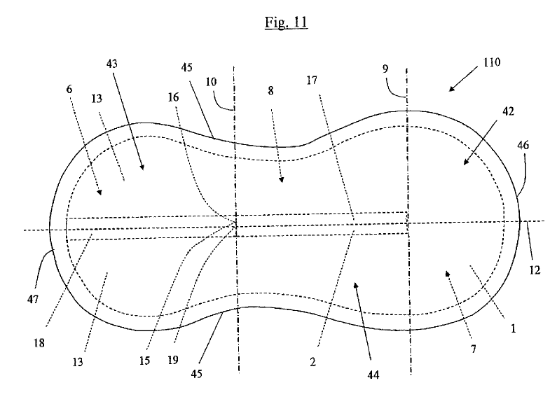

FIG. 11 shows a diagrammatic top view of an absorbent article 11 comprising an absorption sheet 1 according to a fourth embodiment of the invention, where the fold element 2 is in a folded-over postion. The absorbent article 11 here has a shape which corresponds to the shape of the article 110 according to FIG. 10, the difference being that the slits 11 are parallel and the fold element 2 is thus rectangular. Parallel slits are of course possible in an absorption sheet according to the embodiments shown in FIGS. 1-4.

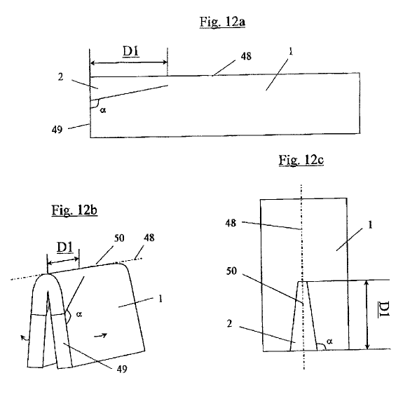

FIGS. 12a-12c are diagrammatic representations of a method for producing an absorption sheet 1 according to the present invention. FIG. 12 shows the absorption sheet 1 as a rectangular sheet folded in two along a longitudinally extending center line 48. After folding it in two, the slits 11 are formed by making a cut through the double-folded absorption sheet 1 along the distance D1. The extent of the slits 11 depends on the angle α between the slits 11 and the folded side 49 of the absorption sheet. When the angle α=0, a parallel slit formation is obtained like that described in connection with FIG. 11. By contrast, when the angle α is greater than zero, the diagonal slits shown in FIGS. 1-10 are obtained.

FIG. 12b shows a perspective view of the absorption sheet 1 from FIG. 12a, the arrows in FIG. 12b indicating the direction in which the double-folded absorption sheet 1 is folded out to a flat state. FIG. 12b shows that the folding out of the absorption sheet 1 has begun in the direction of the arrows. FIG. 12b also shows a fold indication 50 on the double-folded absorption sheet. The fold indication 50 extends along the entire absorption sheet along the longitudinally extending center line 48.

FIG. 12c shows a diagrammatic representation of the absorption sheet 1 according to FIGS. 12a and 12b in a folded-out state after slitting. When the absorption sheet 1 has been folded out to a flat state, the fold element 2 is folded over the absorption sheet in accordance with the described embodiments.

FIGS. 12a-12c show that the double-folded fold element 2 has, by being folded in two, acquired the above-mentioned fold indication 50. If the fold element 2 is not folded back to a flat state at the same time as the rest of the absorption sheet 1, the fold element 2 will be in a semi-folded state when the rest of the absorption sheet is folded out into a flat state. The double-folded fold element 2 can then be folded over the absorption sheet 1 in such a way that the fold indication 50 present in the fold element coincides with the top part of the elevation 17 shown in FIG. 9 as 51. The advantage of this is that it facilitates the process of folding the fold element 2 according, for example, to the embodiment described in FIG. 8.

The invention is not limited to the embodiments shown in the figures and instead can be varied within the scope of the attached patent claims. For example, the central part of the fold element does not need to be rectangular, and instead it can be in the shape of a parallel trapezoid. The elevation can therefore have a triangular (or parallel trapezoid) shape seen in perspective from above when the fold element is folded in between the central part of the fold element and the absorption sheet.

Although only preferred embodiments are specifically illustrated and described herein, it will be appreciated that many modifications and variations of the present invention are possible in light of the above teachings and within the purview of the appended claims without departing from the spirit and intended scope of the invention.

1. An absorbent article, comprising:
a cover sheet, a backsheet and an absorption body disposed therebetween these;
wherein the absorption body comprises an absorption sheet with a longitudinal extent between two short sides and a lateral extent between two long sides, where the absorption sheet is divided, in the longitudinal direction, into a rear portion, a front portion and a central portion extending between the rear portion and the front portion;
an elevation disposed on the absorption sheet, wherein the rear portion is divided, in the lateral direction, into two side portions and, extending between the two side portions, an open intermediate portion formed by cutting out a fold element from the absorption sheet, the fold element constituting a part of the absorption sheet which is folded over itself and thus forms the elevation.

2. The absorbent article according to claim 1, wherein the fold element is folded along a lateral fold line which coincides with a laterally extending first edge of the open intermediate portion.

3. The absorbent article according to claim 2, wherein the fold element is substantially rectangular, and the lateral fold line coincides with a part of the rectangle which constitutes one short side of the rectangle.

4. The absorbent article according to claim 2, wherein the fold element is substantially in the form of a parallel trapezoid comprising a longitudinally extending rectangular central part flanked on both sides by angled parts, and the lateral fold line coincides with a part of the parallel trapezoid which constitutes the shorter of the two parallel sides of the parallel trapezoid.

5. The absorbent article according to claim 2, wherein the fold element is substantially in the form of a parallel trapezoid comprising a longitudinally extending rectangular central part flanked on both sides by angled parts, and the lateral fold line coincides with that part of the parallel trapezoid which constitutes the longer of the two parallel sides of the parallel trapezoid.

6. The absorbent article according to claim 1, wherein the fold element corresponds to the design of the open intermediate portion when the absorption sheet is in the unfolded position.

7. The absorbent article according to claim 4, wherein the elevation includes the central part of the fold element and the angled parts folded in between the absorption sheet and the central part along longitudinally extending parallel fold lines, in such a way that the elevation acquires a substantially rectangular or triangular profile seen in perspective view from above.

8. The absorbent article according to claim 4, wherein the elevation includes the central part of the fold element being folded in two over a centrally positioned longitudinal fold line, and the angled parts of the fold element are unfolded against the absorption sheet substantially at right angles to the double-folded central part, in such a way that the elevation acquires a profile in the form of a cone with brim.

9. The absorbent article according to claim 1, wherein the elevation includes the fold element folded in two on itself along a laterally extending fold line.

10. The absorbent article according to claim 1, wherein the elevation includes the fold element rolled up in its longitudinal extent.

11. The absorbent article according to claim 1, wherein the elevation comprises a stiffening element which strengthens the shape of the elevation.

12. The absorbent article according claim 1, wherein the elevation is formed substantially on the central portion.

13. A method for producing an absorbent article including a cover sheet, a backsheet and an absorption body between the cover sheet and backsheet, where the absorption body comprises an absorption sheet with a longitudinal extent between two short sides and a lateral extent between two long sides, where the absorption sheet is divided, in the longitudinal direction, into a rear portion, a front portion and a central portion extending between the rear portion and the front portion, said method comprising the following steps:
forming a first slit and second slit in the rear portion in such a way that the rear portion is divided, in the lateral direction, into two side portions and, extending between the two side portions, a portion which forms a fold element, and;
folding the fold element over the absorption sheet in such a way that the fold element forms an elevation on the absorption sheet.

14. The method according to claim 13, wherein the fold element is folded over the absorption sheet along a lateral fold line, thus forming the elevation, thereby forming an open intermediate portion between the two side portions, and the lateral fold line coincides with a laterally extending first edge of the open intermediate portion.

15. The method according to claim 14, wherein the steps of forming slits is carried out in two substantially parallel slits from the short side of the rear portion in a substantially longitudinal direction, in such a way that the fold element is substantially rectangular, and the first edge constitutes one of the sides of the fold element.

16. The method according to claim 14, wherein the steps of forming slits is carried out in two substantially diagonal slits from the short side of the rear portion in a substantially longitudinal direction, in such a way that the fold element is substantially in the form of a parallel trapezoid comprising a longitudinally extending rectangular central part flanked on both sides by angled parts, and the first edge constitutes the shorter of the two parallel sides of the parallel trapezoid.

17. The method according to claim 14, wherein the step of forming slits is carried out in two substantially diagonal slits from the short side of the rear portion in a substantially longitudinal direction, in such a way that the fold element is substantially in the form of a parallel trapezoid comprising a longitudinally extending rectangular central part flanked on both sides by angled parts, and the first edge constitutes the longer of the two parallel sides of the parallel trapezoid.

18. The method according to claim 16, wherein the angled parts of the fold element are folded in between the absorption sheet and the central part of the fold element, along longitudinally extending parallel fold lines, in such a way that the elevation acquires a substantially rectangular or triangular profile seen in perspective view from above.

19. The method according to claim 16, wherein the central part of the fold element is folded in two over a centrally positioned longitudinal fold line and the angled parts of the fold element are unfolded across the absorption sheet substantially at right angles to the double-folded central part in such a way that the elevation acquires a profile in the form of a cone with brim.

20. The method according to claim 13, wherein the fold element is folded in two on itself along a laterally extending fold line to form the elevation.

21. The method according to claim 13, wherein the fold element is rolled up in its longitudinal extent to form the elevation.

22. The method according to claim 13, wherein the elevation is formed substantially over the central portion of the absorption sheet.

23. The method according to claim 13, wherein the absorption sheet is folded in two along a longitudinally extending center line, after which the slits are produced by making a cut through the double-folded absorption sheet, after which the absorption sheet is unfolded into a flat state and after which the fold element is folded over the absorption sheet.

24. The method according to claim 23, wherein the double-folded fold element has, by being folded in two, acquired a fold indication, in that the fold element is not folded back to a flat state, but instead the fold element in its substantially double-folded state is folded over the absorption sheet in such a way that the fold indication present in the fold element coincides with the top part of the elevation.
